(19) United States
(12) Patent Application Publication
(10) Pub. No.: US 2023/0042121 A1
(43) Pub. Date: Feb. 9, 2023

(54) METHOD AND SYSTEM FOR MANUFACTURING FORMED PRODUCT

(71) Applicant: YUPO CORPORATION, Tokyo (JP)

(72) Inventors: Manabu WATANABE, Ibaraki (JP); Kou NAKAMURA, Ibaraki (JP); Tsuyoshi DANNO, Ibaraki (JP)

(73) Assignee: YUPO CORPORATION, Tokyo (JP)

(21) Appl. No.: 17/793,508

(22) PCT Filed: Jan. 20, 2021
(86) PCT No.: PCT/JP2021/001925

§ 371 Date: Jul. 18, 2022

(30) Foreign Application Priority Data
Jan. 20, 2020 (JP) 2020-006614

(51) Int. Cl. B29C 48/36; B29C 48/92

(52) U.S. Cl. CPC B29C 48/36; B29C 48/92; G01N 33/44

(57) ABSTRACT

A method for producing a formed product using a resin composition including a plurality of raw materials, in which the content of raw materials in the resin composition is measured in real time, the method comprising the steps of: supplying each of the plurality of raw materials to an extruder; melt-kneading the plurality of raw materials in the extruder to prepare a resin composition; and irradiating the prepared resin composition with radiation and calculating the content of the raw material in the resin composition based on the results of detection of the radiation transmitted through the resin composition.

### TECHNICAL FIELD

The present invention relates to a method and a system for producing a formed product.

### BACKGROUND ART

Porous resin films have been used as printing paper instead of paper made from pulp. These porous resin films are usually produced by forming a resin composition prepared by adding a filler to a thermoplastic resin into film and stretching the film (see, for example, Patent Literatures 1, 2). Fine pores in the film give the porous resin film a texture like paper made from pulp.

### CITATION LIST

### Patent Literature

Patent Literature 1: Japanese Patent Laid-Open No. 9-066564
Patent Literature 2: Japanese Patent Laid-Open No. 2013-010931

### SUMMARY OF INVENTION

### Technical Problem

In a process of producing a resin film, some unmarketable resin film may be discharged. For example, both ends of a resin film fastened with a clip during stretching are cut and discharged. Furthermore, since the composition of a resin film is unstable immediately after the start of production, the resin film produced at an initial stage may be discharged as a non-standard product.

Such discharged products are recovered and reused as a raw material of a resin film to be newly produced from a viewpoint of an environmental and economic protection. In that case the discharged product recovered (hereinafter referred to as a recovered raw material) is supplied to the manufacturing line together with each raw material to be newly added, such as a thermoplastic resin and a filler, to prepare a resin composition. Since the filler component has a great impact on properties of a resin film, it is necessary to measure the content of the filler in the resin composition and determine the amount of each raw material to be supplied so that the content of the filler becomes constant in order to maintain a certain quality of the resin film.

The content of the filler component in the resin composition may be determined by collecting a sample in a system and measuring the filler component which remains after baking the resin component in the sample (which may be referred to as ash content or ash). However, the method takes time and thus the measurement becomes intermittent. The content of the filler component in the resin film to be produced may fluctuate during measurement, and the amount of each raw material supplied cannot be feedback-controlled in real time. Thus, it is difficult to ensure the quality of resin films continuously produced.

An object of the present invention is to measure the content of raw materials in a resin composition in real time.

### Solution to Problem

The present inventors have conducted intensive studies to solve the above problem and as a result have found that the problem can be solved by a measurement using radiation, and have completed the present invention.

Accordingly, the present invention is as follows.

(1) A method for producing a formed product using a resin composition comprising a plurality of raw materials, the method comprising the steps of:

supplying each of the plurality of raw materials to an extruder;

melt-kneading the plurality of raw materials in the extruder to prepare a resin composition; and

irradiating the prepared resin composition with radiation and calculating the content of the raw material in the resin composition based on the results of detection of the radiation transmitted through the resin composition.

(2) The method according to (1) above, wherein

the plurality of raw materials comprise at least a thermoplastic resin and a filler, and

the step of calculating the content comprises a step of calculating the content of the filler in the resin composition.

(3) The method according to (2) above, wherein the filler is an inorganic filler.

(4) The method according to any of (1) to (3) above, wherein the step of calculating the content comprises

a step of irradiating the resin composition in a molten state with radiation and detecting the radiation transmitted through the resin composition.

(5) The method according to any of (1) to (4) above, wherein the step of calculating the content comprises

a step of calculating the density of the resin composition from the results of detection of the radiation and

a step of calculating the content of the raw material based on the calculated density of the resin composition and the density of each of the raw materials in the resin composition.

(6) The method according to any of (1) to (5) above, wherein the step of calculating the content comprises calculating the content of the raw material based on at least one condition of the temperature and the pressure of the resin composition upon irradiation with the radiation.

(7) The method according to any of (1) to (6) above, wherein the radiation is an X ray or a γ ray.

(8) The method according to any of (1) to (7) above, wherein the formed product is continuously produced.

(9) The method according to any of (1) to (8) above, wherein part of the plurality of raw materials supplied to the extruder is supplied to the extruder in the form of a mixture.

(10) The method according to any of (1) to (9) above, wherein the mixture is a recovered raw material comprising the whole or part of the formed product discharged in a process of producing the formed product.

(11) The method according to any of (1) to (10) above, comprising a step of calculating the content of a raw material in the recovered raw material supplied to the extruder based on the content of the raw material in the resin composition and the amount of each of the raw materials supplied.

(12) The method according to any of (1) to (11) above, wherein the formed product is a film or a pellet.

(13) The method according to any of (1) to (12) above, comprising

a step of controlling the amount of each of the raw materials supplied to the extruder based on the calculated content of the raw material.

(14) A system for producing a formed product using a resin composition comprising a plurality of raw materials, the system comprising:

an extruder for melt-kneading the plurality of raw materials to prepare a resin composition;

a measuring device for irradiating the prepared resin composition with radiation and measuring the results of detection of the radiation transmitted through the resin composition; and

a calculator for calculating the content of the raw material in the resin composition based on the results of detection.

(15) The system according to (14) above, comprising a controller for controlling the amount of each of the raw materials supplied to the extruder based on the calculated content of the raw material.

### Advantageous Effect of Invention

The present invention can measure the content of a raw material in a resin composition in real time.

### BRIEF DESCRIPTION OF DRAWINGS

FIG. 1 is a schematic view illustrating an example of the structure of a system for producing a single layer film.

FIG. 2 is a flowchart showing the feedback control in the first embodiment.

FIG. 3 is a graph showing an example of a first calibration curve.

FIG. 4 is a graph showing an example of a second calibration curve.

FIG. 5 is a graph showing examples of corrected second calibration curves.

FIG. 6 is a schematic view illustrating an example of the structure of a system for producing a laminated film.

FIG. 7 is a flowchart showing the feedback control in the second embodiment.

FIG. 8 is a table illustrating the composition of a recovered raw material derived from a laminated film.

### DESCRIPTION OF EMBODIMENTS

The method and system for producing a formed product of the present invention will be described in detail below. The following describes an example (a typical example) of the present invention and the present invention is not limited thereto.

The method for producing a formed product of the present invention is a method for producing a formed product using a resin composition comprising a plurality of raw materials. The method for producing a formed product of the present invention comprises the steps of: supplying the plurality of raw materials to an extruder; melt-kneading the plurality of raw materials in the extruder to prepare a resin composition; and irradiating the prepared resin composition with radiation and calculating the content of the raw material in the resin composition based on the results of detection of radiation transmitted through the resin composition.

More specifically, the content of a specific raw material is calculated from the density of the specific raw material which has been previously found, the density of all raw materials other than the specific raw material and the density of a resin composition, which is determined based on the results of detection of the radiation, and the calculated content is used in the method for producing a formed product. It is preferable that the difference between the density of the specific raw material and the density of all raw materials other than the specific raw material is large because the present invention can be easily applied.

In particular, when producing a formed product such as film using at least a thermoplastic resin and a filler as a raw material, it is preferable to calculate the content of the filler as the specific raw material. This is because while the quality of a formed product is likely to vary when the content of the filler fluctuates, the amount of each raw material supplied can be controlled in real time so that the content of the filler in the resin composition falls within a certain range based on the content of the filler calculated.

A discharged product of a formed product, a non-standard product generated unexpectedly (referred to as an off-specification product) or a recovered raw material recovered from another system may be used as a raw material for the formed product together with new raw materials freshly supplied. At that stage, in the method of the present invention, a resin composition in a molten state transferred in an extruder is irradiated with radiation, and the density of the resin composition is calculated from the results of detection of radiation transmitted through the resin composition, and the content of the filler in the resin composition can be calculated from the density.

While the raw materials of the formed product are weighed and then supplied to the extruder, the amount of each of the components of the formed product does not always reach the target value and may fluctuate depending on the condition of manufacture or accuracy of measurement in the manufacture. Thus, the content of the filler in the formed product may fluctuate even when only a new raw material which is freshly supplied is used, and the content is more likely to fluctuate when a recovered raw material is used in combination.

In this way, even when the content of the filler fluctuates, the method of the present invention can stabilize the quality by calculating the content of the filler in the resin composition in a molten state before forming. The amount of each of the raw materials supplied can be easily feedback-controlled based on the calculated content so that the content of the filler in the formed product reaches the target value. Filler is added as a nucleating agent which forms pores in a formed product or as a pigment which does not form pores but increases the degree of whiteness, and is an important component which determines properties of the formed product such as whiteness and mechanical strength. Although fluctuation of the content of filler makes the quality of the formed product unstable, the above feedback control ensures a certain quality for long time.

### First Embodiment

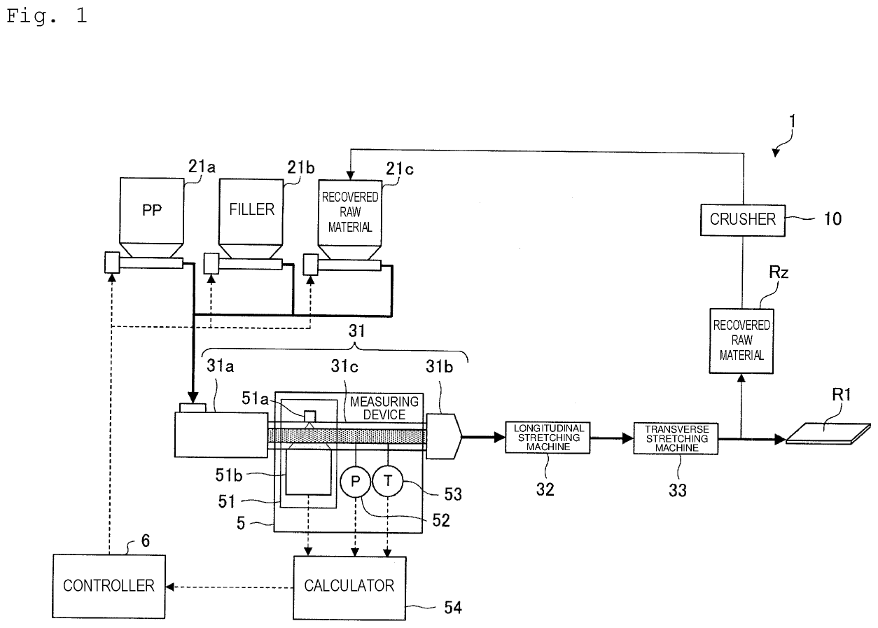

FIG. 1 shows an example of a system for production to which the method of the present invention is applied.

In the system for production 1 shown in FIG. 1, a single layer resin film R1 is produced by forming a resin composition prepared by mixing a filler to a thermoplastic resin.

### (Raw Materials of Formed Product)

### <Thermoplastic Resin>

The thermoplastic resin, which is a raw material of resin film R1, is not particularly limited. A polyolefin-type resin such as polypropylene, polyethylene, polybutene and a 4-methyl-1-pentene (co)polymer is preferred, and in particular, polypropylene and polyethylene are more preferred from the viewpoint of formability and mechanical strength of the film. One thermoplastic resin may be used alone or two or more of them may be used in combination. From the viewpoint of formability of pores, using polyethylene together with polypropylene is preferred. When two or more thermoplastic resins are used as described above, it is preferable that the difference in the density of each of the resins is small.

### <Filler>

Examples of fillers include inorganic fillers and organic fillers, and they may be used alone or in combination. Stretching of a resin composition containing a filler allows many fine pores to be formed in the film or on the surface with the filler as a nucleus, making the resin film R1 white, opaque and lightweight. This also provides the resin film R1 with a paper-like texture. When a filler is included as a pigment, whiteness of the film can be increased without pores.

Inorganic fillers are preferred from the viewpoint of formability for pores and the cost. Inorganic fillers are preferred also because difference in the density with the thermoplastic resin is large and thus the content of the filler can be accurately calculated.

Examples of inorganic fillers include heavy calcium carbonate, light calcium carbonate, titanium oxide, baked clay, talc and inorganic particles prepared by surface treatment of them with fatty acid, a polymer surfactant, an anti-static agent or the like. One of the above may be used alone or two or more of them may be used in combination. In particular, heavy calcium carbonate and light calcium carbonate are preferred from the viewpoint of the difference in the density with the thermoplastic resin and the cost. When two or more inorganic fillers are used, it is preferable that the difference in the density of the fillers is small.

The filler has an average particle size of preferably 0.01 μm or more, more preferably 0.1 μm or more, and further preferably 0.5 μm or more. The filler has an average particle size of preferably 30 μm or less, more preferably 20 μm or less, and further preferably 15 μm or less. When the average particle size is the lower limit or more, the filler is easily mixed with the thermoplastic resin. When the average particle size is the upper limit or less, sheet is unlikely to be broken in stretching and the strength of film is less likely to be reduced.

For the average particle size of the filler, a section of film is observed with an electron microscope and an average value in the measurement of the maximum diameter of at least 10 particles when the filler is dispersed in a thermoplastic resin by melt-kneading is determined as an average dispersion particle size.

The content of the filler in the resin composition is preferably 80% by mass or less, more preferably 60% by mass or less, and further preferably 50% by mass or less from the viewpoint of accuracy of the results of measurement of radiation. The content is 3% by mass or more, more preferably 5% by mass or more, and further preferably 10% by mass or more. When the content is the upper limit or less, the filler is less likely to absorb or scatter radiation excessively, and thus the intensity of radiation is unlikely to be reduced and accuracy of detection is easily improved. When the content is the lower limit or more, absorption and scattering of radiation is moderate, and accuracy of detection of radiation is easily increased.

The content of the filler in the resin composition is preferably 1% by mass or more, more preferably 3% by mass or more, and further preferably 5% by mass or more from the viewpoint of making the film opaque. The content is preferably 65% by mass or less, more preferably 50% by mass or less, and further preferably 40% by mass or less from the viewpoint of making the film rigid to improve handling properties.

The content of the filler in the resin composition when the filler is used as a pigment is preferably 0.1% by mass or more, and more preferably 0.3% by mass or more. The content is preferably 20% by mass or less, and more preferably 10% by mass or less. When the content is the above lower limit or more and the upper limit or less, a moderate whiteness and opaqueness are easily given to the film. Titanium oxide is preferred when the filler is used as a pigment.

### (System for Producing Formed Product)

The system for production 1 has meters 21a to 21c, an extruder 31, a longitudinal stretching machine 32, a transverse stretching machine 33 and a crusher 10.

Meters 21a to 21c usually have a hopper for introducing raw materials, a feeder for feeding the raw material weighed to the extruder 31 from the hopper and a driving member (e.g., a motor) for opening and closing a valve provided in an opening of the hopper and for driving the feeder.

The meter 21a supplies polypropylene (PP) and the meter 21b supplies filler. They are new raw materials which have a single component and are freshly supplied thereto to produce resin film R1. The meter 21c supplies recovered raw material Rz recovered from resin film R1. The new raw material may be not only a raw material having a single component but also pellets prepared by mixing a plurality of components (what is called master batch pellets).

When only new raw materials are used, production of resin film R1 is conducted as follows.

### <Step for Supplying Raw Material>

First, each of the raw materials is weighed in meters 21a to 21c and supplied to the extruder 31. The system for production 1 may have a mixer between the meters 21a to 21c and the extruder 31, and each of the raw materials may be mixed in the mixer and then supplied to the extruder 31. The system may also have a hopper between the meters 21a to 21c and the extruder 31 or between the meters 21a to 21c and the mixer.

### <Step for Preparing Resin Composition>

Each of the raw materials supplied to the extruder 31 is melt-kneaded in a screw unit 31a of the extruder 31 to prepare a resin composition containing each of the raw materials.

### <Step of Forming>

The resin composition in a molten state passes through pipe 31c and is extruded into sheet through a die 31b arranged at the end of the extruder 31. The melting temperature for the resin composition may be determined based on the melting point of the resin to be used and its viscosity in the molten state. The melting temperature is usually 70 to 300° C. and is about 70 to 280° C. when the thermoplastic resin is a polyolefin-type resin.

### <Step of Stretching>

The non-stretched film is stretched in the machine direction (MD) in the longitudinal stretching machine 32 and stretched in the transverse direction (TD) in the transverse stretching machine 33.

Examples of stretching methods include a longitudinal stretching method in which the difference in the peripheral speed of a group of rolls is used, a transverse stretching method using a tenter oven, a sequential biaxial stretching method combining the above, a rolling method, a simultaneous biaxial stretching method in which a tenter oven and a pantograph are combined, and a simultaneous biaxial stretching method in which a tenter oven and a linear motor are combined. A biaxial stretching method (inflation forming) in which the molten resin is extruded in the form of tube using a circular die connected to a screw extruder and then air is blown into it may also be used.

Finally, the film is finished by, for example, cutting both ends of the film in the transverse direction to give a product, i.e., a single layer resin film R1. The film may also be coated with a coating solution by a coater after stretching and dried by a dryer to give a resin film R1. The coating layer is formed, for example, in order to improve printing properties. An embossing treatment and the like may also be performed as needed.

In the above process of production, a discharged product of resin film R1 may be generated. Discharged products include a resin film which has been regarded as a non-standard product in a regular inspection (may also be referred to as an off-specification product), a resin film which has been produced immediately after the start of production under conditions in which the composition is unstable and both ends of a resin film fastened with a clip during stretching and cut.

These discharged products are recovered and crushed into chips in a crusher 10 and then supplied to the hopper of the meter 21c as a recovered raw material Rz. The recovered raw material Rz supplied to the meter 21c is reused as one of the plurality of raw materials of the resin film R1 to be newly produced. The recovered raw material Rz is discharged in the process of production and is made of the resin film R1. In other words, the recovered raw material Rz is a mixture of a thermoplastic resin, which is a raw material of the resin film R1, and a filler.

### <Feedback Control>

The system for production 1 of the present embodiment has a measuring device 5, a calculator 54 and a controller 6 so as to feedback control the amount of each raw material supplied so that the content of the filler in the resin film R1 becomes constant.

The measuring device 5 has a detecting member 51, a pressure gauge 52 and a thermometer 53 as shown in FIG. 1. The detecting member 51 has a radiation source 51a and a detector 51b.

The detecting member 51 irradiates the resin composition in pipe 31c of the extruder 31 with radiation from the radiation source 51a. Examples of radiations with which the resin composition is irradiated include X rays, β rays and γ rays. X rays and γ rays are preferred from the viewpoint of transmissivity through pipe 31c. γ rays are more preferred from the viewpoint of miniaturization of the device.

Although the type of radiation sources is not particularly limited, examples thereof include Na-22, Co-57, Co-60, Ba-133 and Cs-137. A suitable radiation source may be selected depending on the purpose of use and situations. Ba-133 is preferred from the viewpoint of the intensity of radiation, the life of the radiation source and easy handling.

The intensity of radiation is preferably 1 MBq or more, and more preferably 5 MBq or more. The intensity of radiation is preferably 50 MBq or less, and more preferably 20 MBq or less. When the intensity of radiation is in the above range, the content of raw materials can be measured at high accuracy.

In the detecting member 51, the detector 51b arranged at a position opposed to the radiation source 51a with the resin composition in the pipe 31c therebetween detects the radiation transmitted through the resin composition. The detector 51b, for example, is a scintillation counter.

The pressure gauge 52 measures the pressure of the resin composition in the pipe 31c. The thermometer 53 measures the temperature of the resin composition in the pipe 31c. It is preferable that the position of measurement by the pressure gauge 52 and the thermometer 53 is near the position where the resin composition is irradiated with radiation from the detecting member 51.

The calculator 54 calculates the content of raw materials in the resin composition based on the results of detection of irradiation by the detecting member 51.

The controller 6 controls each of the meters 21a to 21c based on the difference between the calculated value of the content of raw materials, for example, a filler, calculated by the measuring device 5 and the target value to adjust the amount of each of the raw materials supplied to the extruder 31. For the control of the amount to be supplied, the amount may be controlled each time when a difference arises between the calculated value and the target value (set value), or the amount may not be controlled immediately after a difference arises but may be controlled when the difference goes out of tolerance. For example, when the calculated value exceeds the target value (set value), the controller 6 can reduce the amount of the filler to be supplied, increase the amount of the thermoplastic resin to be supplied, or both, depending on how large the difference.

A computer or a microcomputer having a processor such as a CPU (central processing unit) and a memory may be used as the calculator 54 and the controller 6.

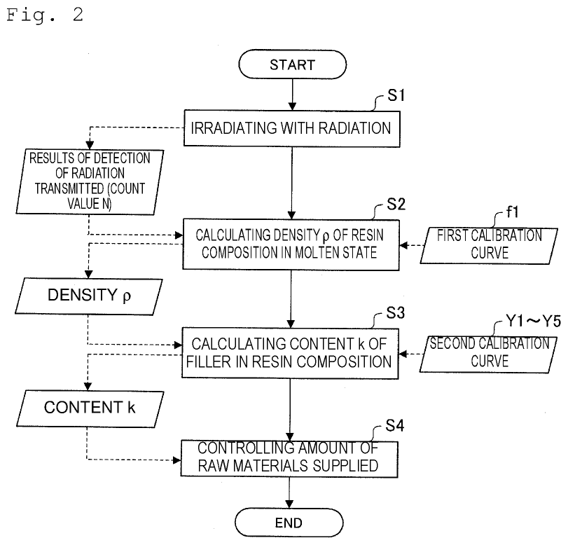

FIG. 2 shows a processing flow of feedback control in the system for production 1. The process is repeated at regular intervals in the production of the resin film R1. By setting the interval to be short, smoother control in real time is enabled.

### <<Step for Detecting Radiation>>

In feedback control, the resin composition in a molten state in pipe 31c is irradiated with radiation by means of detecting member 51. The detecting member 51 detects the amount of radiation transmitted through the resin composition, and then the calculator 54 receives the results of detection from the detecting member 51 (Step S1).

### <<Step for Calculating Density>>

The calculator 54 calculates the density of the resin composition based on the results of detection of radiation by the detecting member 51 (Step S2). When the resin composition is irradiated with radiation, the resin composition absorbs or scatters radiation. Since the amount of radiation transmitted through the resin composition and the density of the resin composition are co-related, the density of the resin composition can be calculated from the results of detection of radiation transmitted through the resin composition.

More specifically, the calculator 54 calculates the density ρ (g/cm³) of the resin composition from the count value N (cps) detected in the detector 51b based on the first calibration curve f1 showing the correlation between the count value N of (cps) of radiation and the density ρ (g/cm³) of the resin composition.

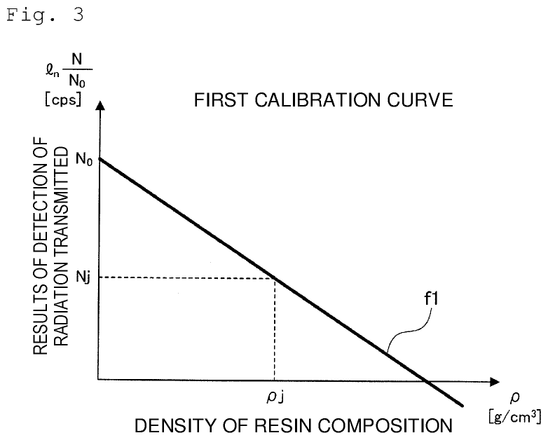

FIG. 3 illustrates an example of the first calibration curve f1.

The first calibration curve f1 illustrated in FIG. 3 shows a correlation between ln(N/N₀)(cps) determined form the count value N of γ rays transmitted through the resin composition and the density (g/cm³) of the resin composition. When the count value of γ rays is Nj, the calculator calculates the density ρ of the resin composition as ρj based on the calibration curve f1.

The above first calibration curve f1 can be previously experimentally determined. More specifically, an empty pipe 31c in which no resin composition is put and a pipe 31c in which a resin composition with a known density is put are irradiated with radiation from the detecting member 51, and γ rays transmitted through the pipe are detected, respectively. The following expression (1) shows a correlation between the density ρ of the resin composition and the count value N₀ in the empty state and between the density ρ and the count value N in the packed state, and thus the following expression (2), which indicates the first calibration curve f1, can be derived.

$$\begin{matrix}[ Expression 1 ] & \\ N = {N}_{0} {e}^{- μ ρ} & ( 1 )\end{matrix}$$

$$ln ( \frac{N}{{N}_{0}} ) = - μ ρ \qquad ( 2 )$$

ρ: Density of target of measurement

μ: Device constant

### <<Step for Calculating Amount of Filler>>

Next, the calculator 54 calculates the content of the filler (% by mass) in the resin composition based on the density ρ of the resin composition and the density of each of the raw materials calculated (Step S3). For the density of each of the raw materials, a known density or a measured density may be saved in the calculator 54.

The content k of the filler may be calculated based on the density ρ of the resin composition and the density of each of the raw materials calculated. An example of calculation will be described, in which the density of the thermoplastic resin is 0.9 g/cm³, the density of the filler is 0.5 g/cm³, and the density of the thermoplastic resin calculated after the irradiation of radiation is 0.75 g/cm³. If the content of the thermoplastic resin is assumed as x % by mass and the content of the filler is assumed as y % by mass, the following two equations are established. These simultaneous equations yield x=75, y=25.

x+y=100

100/{(x/0.9)+(y/0.5)}=0.75

By subtracting the amount supplied (% by mass) from the meter 21a and the meter 21b from the content (% by mass) of the thermoplastic resin and the filler determined above, respectively, the ratio of the content of the thermoplastic resin and the filler in the recovered raw material Rz can be calculated.

Although the calculator 54 may perform calculations each time as described above, calculation is much easier when the second calibration curve Y1 is previously prepared and used. The second calibration curve Y1 is previously obtained from the density of the resin composition and the content of filler therein and saved in the calculator 54. More specifically, the second calibration curve Y1 may be prepared by determining the density of the resin composition when the content of the filler in the resin composition is changed.

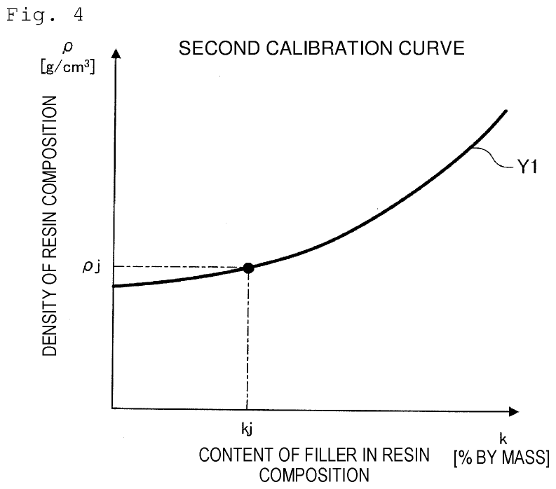

FIG. 4 illustrates an example of the second calibration curve Y1. The second calibration curve Y1 shows a correlation between the density of a resin composition (g/cm³) composed of a thermoplastic resin and a filler and the content of the filler (% by mass) in the resin composition. The density of the resin composition refers to a density of the resin composition at a typical temperature (° C.) and pressure (MPa).

The density of a thermoplastic resin fluctuates depending on the condition of temperature and pressure, and thus the correlation between the density of the resin composition and the content of the target raw material in the resin composition also fluctuates depending on the condition of temperature and pressure. From the viewpoint of improvement of accuracy of calculation of the content of raw materials, it is preferable that the calculator 54 calculates the content of raw materials based on at least one condition of the temperature and the pressure of the resin composition when the composition is irradiated with radiation. It is preferable that the calculator 54 calculates the content of raw materials based on the temperature of the resin composition when the composition is irradiated with radiation because the condition of temperature causes large fluctuation.

More specifically, of the second calibration curves prepared in environments in which conditions of temperature and pressure of a resin composition are different, a second calibration curve prepared under a condition corresponding to the condition of temperature and pressure when the composition is irradiated with radiation is used. Alternatively, in the calculator 54, a second calibration curve prepared at a predetermined temperature and pressure may be used in usual cases, and when the temperature and the pressure measured are different from those previously set, the second calibration curve usually used may be corrected to a second calibration curve corresponding to the temperature and the pressure.

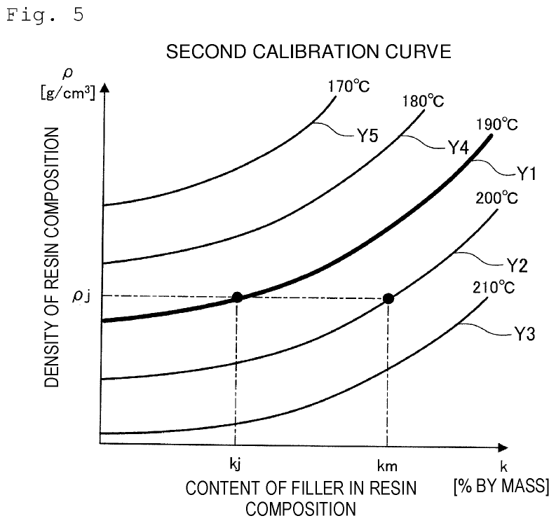

FIG. 5 shows corrected examples of the second calibration curve Y1.

The second calibration curve Y1 is prepared using a resin composition at a temperature of 190° C. The calibration curves Y2 and Y3 are prepared using a resin composition at a temperature of 200° C. and 210° C., respectively. The calibration curves Y4 and Y5 are prepared using a resin composition at a temperature of 180° C. and 170° C., respectively.

For example, when the temperature read by the thermometer 53 is 190° C. and the density of the resin composition calculated is ρj (g/cm³), the calculator produces a content of the filler of kj (% by mass) from the second calibration curve Y1. Meanwhile, when the temperature measured is 200° C., the calculator 54 produces km (% by mass) from the second calibration curve Y2 although the density of the resin composition is the same ρj (g/cm³).

The calculator 54 can calculate the content of each raw material other than the filler based on the ratio of the content of the raw material in the resin composition. When the resin composition is made of two raw materials of polypropylene and a filler as described above and the content of the filler is calculated to be 40% by mass, the content of polypropylene is 60% by mass.

### <<Step for Controlling Amount of Raw Material Supplied>>

When the content of the filler is calculated, the controller 6 controls the meters 21a to 21c to adjust the content of each of the raw materials supplied based on the difference between the calculated value and the target value so that the content of the filler reaches the target value (step S4).

The controller 6 may control any of the amount of a recovered raw material Rz supplied from the meter 21c and the amount of the raw material supplied from the meters 21a and 21b. It is preferable that the controller 6 keeps the amount of the recovered raw material Rz supplied constant to control the amount of raw materials having a single component from the viewpoint of easy control and a stable composition of the raw materials supplied to the extruder 31. Such feedback control is particularly effective when the ratio of mixing of the recovered raw material in the resin composition is high.

The amount of the recovered raw material Rz supplied in the resin composition is preferably 60% by mass or less, and more preferably 50% by mass or less from the viewpoint of ensuring quality.

As described above, according to the first embodiment, the resin composition in a molten state in the extruder 31 is irradiated with radiation and the content of the filler is calculated based on the results of detection of radiation transmitted through the resin composition using the first calibration curve f1 and the second calibration curve f2.

This enables the content of raw materials in a resin composition in the process of producing a resin film R1 to be measured in real time. Since the amount of each raw material supplied can be feedback controlled based on the calculated content, the content of the filler in the resin film R1 can be kept in a certain range even when a recovered raw material Rz is used. Furthermore, sometimes the production of resin film R1 may be once stopped and newly started to produce a different rot of resin film R1, not the same lot of resin film R1. Even in such cases, variation in the quality of resin film R1 caused by a filler can be reduced also for different lots, and thus a certain quality of resin film R1 can be ensured for long time.

Although the content of a filler in a resin composition may be determined by weighing the filler component remaining after baking a resin component in the resin composition (which may be a recovered raw material Rz in the case where the resin composition to be measured is the same as the recovered raw material as with the case of a monolayer resin film R1), weighing takes time. According to the present embodiment, it does not take long to calculate the content of a filler and thus the amount of the filler supplied can be quickly feedback controlled, for example, in a minute, substantially enabling real time control.

Thus, the time in which the ratio of mixing of raw materials fluctuates is significantly reduced and the amount of a resin film R1 with poor quality is reduced, and therefore production loss becomes small. Furthermore, since a recovered raw material Rz discharged in the process of producing a resin film R1 can be consumed in the same production line of the resin film R1, resin films can be produced efficiently. Easy reuse of the recovered raw material Rz improves the total yield.

### Second Embodiment

While a single layer film R1 was produced in the first embodiment, the present invention can be suitably applied to the production of a laminated film having a multi-layer structure. The present invention can also be applied to cases in which a filler is mixed to two or more thermoplastic resins. The second embodiment illustrates such examples of multilayers and multiple components.

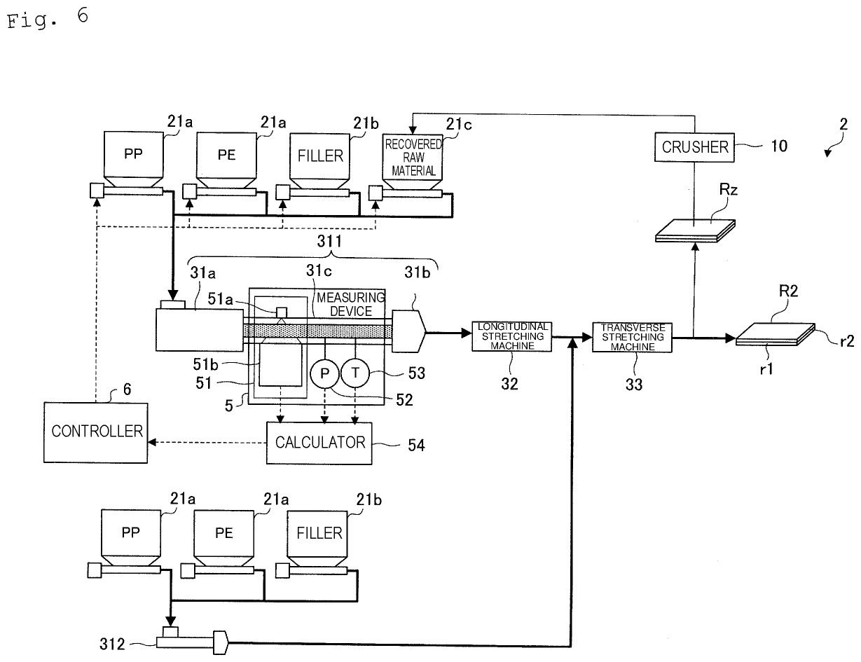

FIG. 6 shows a structure of the system for production 2 of the second embodiment. In the system for production 2, a resin film R2 having a two-layer structure is produced by forming a substrate layer r1 and a surface layer r2 individually and laminating the surface layer r2 on the substrate layer r1. In FIG. 6, the same symbol is used for the same element as in the system for production 1.

The substrate layer r1 and the surface layer r2 are formed by melt-kneading a resin composition prepared by mixing a filler to a thermoplastic resin and extruding it in the form of a sheet. For both, polypropylene (PP) and polyethylene (PE) are used as the thermoplastic resin. The amount of each raw material mixed in each of the films may be the same or different.

In the system for production 2, first, raw materials of the substrate layer r1 are supplied to the extruder 311 from each of the meters 21a to 21c. They are melt-kneaded in the extruder 311 to prepare a resin composition for the substrate layer. The resin composition is extruded in the form of a sheet to form a substrate layer r1. Meanwhile, raw materials of the surface layer r2 supplied from different meters 21a to 21c are melt-kneaded in another extruder 312 to prepare a resin composition for the surface layer. The resin composition is extruded in the form of a sheet to form a surface layer r2.

The substrate layer r1 is stretched in the machine direction in the longitudinal stretching machine 32 and the surface layer r2 is laminated on one side thereof. The laminate is stretched in the transverse direction in the transverse stretching machine 33 to form a two-layer laminated film R2. A coating layer may be formed on the surface of the laminated film R2 as with the case of the monolayer resin film R1.

Unnecessary laminated film R2 may be recovered and used for forming a substrate layer r1 as a recovered raw material Rz also in the system for production 2. The system for production 2 is also equipped with a measuring device 5, a calculator 54 and a controller 6 as in the system for production 1. More specifically, a detecting member 51 is arranged in pipe 31c of the extruder 311 and the detecting member 51 detects the amount of radiation transmitted through the resin composition of the substrate layer r1 when the resin composition is irradiated with radiation.

The calculator 54 calculates the content of filler k (% by mass) in the resin composition of the substrate layer r1 based on the results of detection of the amount of radiation transmitted therethrough as in the first embodiment. The controller 6 feedback-controls the amount supplied of raw materials of the substrate layer r1 based on the difference between the calculated value and the target value of the content.

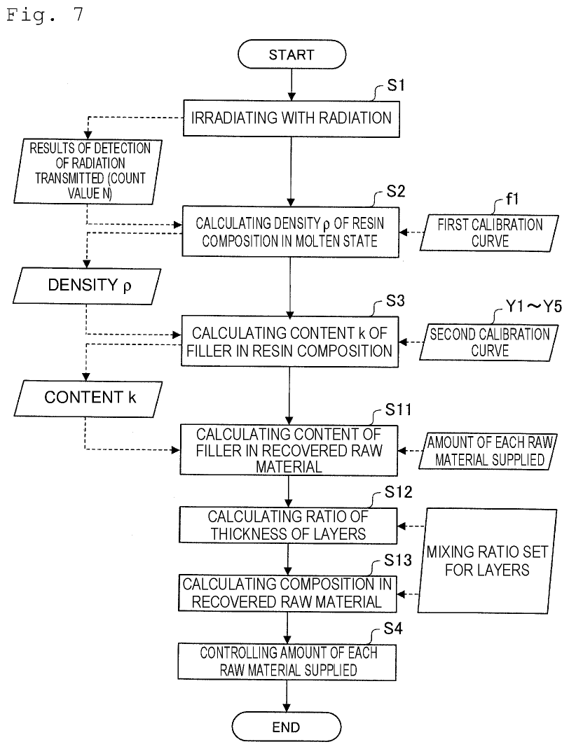

FIG. 7 shows a processing flow of feedback control in the system for production 2.

In the feedback control, the resin composition for the substrate layer is irradiated with radiation by means of the detecting member 51. The detecting member 51 detects the amount of radiation transmitted through the resin composition, and then the calculator 54 receives the results of detection (count value N) from the detecting member 51 (Step S1).

Next, the calculator 54 calculates the density ρ (g/cm³) of the resin composition based on the results of detection of the amount of radiation transmitted therethrough using the first calibration curve f1 (Step S2). The calculator 54 calculates the content k of the filler (% by mass) in the resin composition based on the density ρ of the resin composition calculated using the second calibration curves Y1 to Y5 (Step S3). The above calculation can be performed as in the first embodiment, and thus its detailed explanation will be omitted.

The controller 6 can control the amount of each of the raw materials supplied based on the content k (% by mass) of the filler calculated. In the second embodiment, it is necessary that the calculator 54 first calculates the content of the filler in a recovered raw material Rz and calculates the amount of each raw material supplied based on the calculated value. This is because when the content of each of the raw materials in the recovered raw material Rz is found, the amount of each of the raw materials supplied necessary to control the content of each of the raw materials to the target value can be determined, and the amount supplied can be thus controlled.

The content of the filler in the recovered raw material Rz may be calculated based on the content k of the filler in the resin composition calculated from density ρ and the amount of each of the raw materials supplied from the meters 21a to 21c.

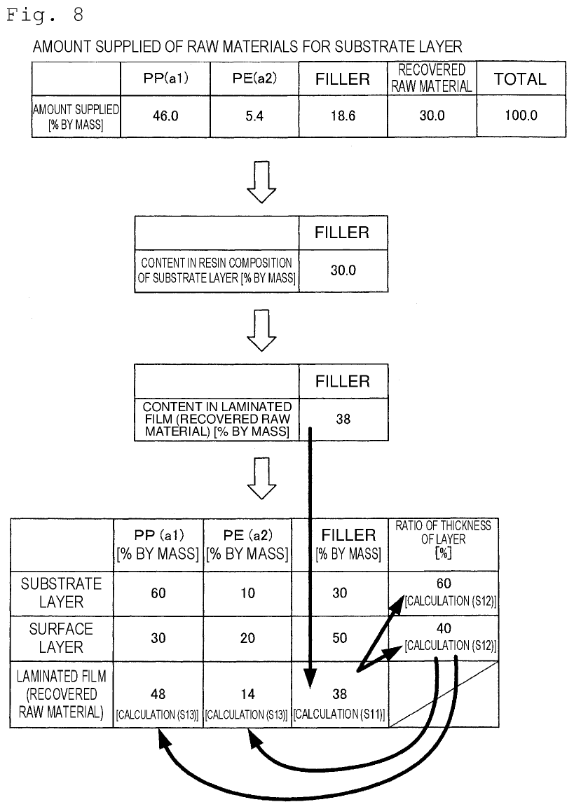

An example of calculating the amount of raw materials of each of the layers of a two-layer laminated film R2 having a substrate layer r1 and a surface layer r2 will be described with reference to FIG. 8. In this Example, the mass ratio (a1:a2:b) among polypropylene (a1), polyethylene (a2) and filler (b), which is the ratio of mixing of each raw material previously set, is 60:10:30 for the substrate layer r1 and 30:20:50 for the surface layer r2. The ratio of mixing is previously set for every grade of the product to be produced. The mass ratio of each of the raw materials of the substrate layer r1 supplied from each of the meters 21a to 21c (a1:a2:b) is 46.0:5.4:18.6:30.0.

When the content of the filler (b) in the resin composition in the pipe 31c is 30% by mass, the ratio of each of the raw materials of the surface layer r1 is (a1:a2:b)=(46.0:5.4:18.6:30.0) as described above when calculated after irradiation of radiation. Thus, (30−18.6)+30×100=38% by mass is given, and the content of the filler in the recovered raw material is found to be 38% by mass. When the content of the resin composition in the pipe 31c derived from the substrate layer r1 is assumed as W1 (% by mass) and the content of the resin composition in the pipe 31c derived from the surface layer r2 is assumed as W2 (% by mass), the following two expressions are established. The calculator 54 calculates W1=60, W2=40 from these simultaneous equations (Step S11).

W1+W2=100 (Whole laminated film R2)

30×W1+50×W2=38×100 (Filler in laminated film R2)

When the ratio of the thickness of the substrate layer r1 to the total thickness of the laminated film R2 is represented by d1 (%), and the ratio of the thickness of the surface layer r2 thereto is represented by d2 (%), the calculator 54 gives d1=60, d2=40 based on the contents W1 and W2 calculated (Step S12).

Next, the calculator 54 calculates the composition of the recovered raw material Rz, i.e., the content of each of the raw materials, based on the ratio of the thickness of each of the films, d1 and d2 calculated and the ratio of mixing of each raw material which is previously set in each of the layers (Step S13). As shown in FIG. 8, this gives a content of polypropylene (a1) in the recovered raw material Rz in each of the films of 48% by mass, a content of polyethylene (a2) of 14% by mass, and a content of the filler (b) of 38% by mass.

When the content of each of the components in the recovered raw material Rz is calculated as described above, the controller 6 controls the meters 21a to 21c so that the content of the filler reaches the target value to adjust the content of each of the raw materials (Step S4). The controller controls them as in the first embodiment, and thus its detailed explanation will be omitted.

As described above, even when the recovered raw material Rz recovered from a laminated film R2 having a multilayer structure is used as a raw material for a substrate layer r1, which is one of the layers of the laminated film R2, the content of the raw material in the resin composition can be measured in real time and feedback control can be performed as in the first embodiment. In the case of films having such a multilayer structure, calculation of the content of the filler in the recovered raw material Rz makes it easier to find a balance between each raw material and thus improves accuracy of control. Although a device for melting the recovered raw material Rz and a measuring device 5 for measuring its density may be separately provided in order to calculate the content of the filler in the recovered raw material Rz, the content of the filler can be found by the above calculation without such effort.

When the content of the filler in the substrate layer r1 is different from that in the surface layer r2 and a recovered raw material Rz recovered from a multilayer laminated film R2 is used as a raw material of the substrate layer r1, the content of the filler in the substrate layer r1 fluctuates. However, the above real time measurement of the content of the filler and feedback control enables a resin film R2 with a certain quality to be produced in a stable manner for a long time even when a resin film R2 having a multilayer structure is used as the recovered raw material Rz.

Furthermore, the content of the filler can be calculated even when two components of polypropylene and polyethylene are used as the thermoplastic resin. This is because the difference in the density is large between the resin component and the filler component constituting the thermoplastic resin. A large difference in the density enables the content of filler to be calculated more accurately. Polypropylene and polyethylene are both a polyolefin-type resin, and the whole resin component has substantially the same density, and thus the difference in the density with that of the filler is as large as a one component resin, and therefore the content of the filler can be calculated accurately. Furthermore, the change in the density of the whole resin component is small when different types of polyolefin-type resins are used. Thus, the content of the filler in the resin composition can be accurately calculated even when a polyolefin-type resin other than polypropylene or polyethylene is used, or two or more polyolefin-type resins are used.

When the type of the thermoplastic resin used for the surface layer r2 is different from that for the substrate layer r1, a resin originally not used for the substrate layer r1 is mixed in the substrate layer r1 if a recovered raw material Rz is used. However, the impact of the thermoplastic resin is smaller than the impact of the filler on the quality of the resin film, and the difference in the type of the thermoplastic resin has little impact on the quality. Furthermore, when the amount of mixing of the recovered raw material Rz is 50% by mass or less, there is little fluctuation in quality. When the resin to be used is a thermoplastic resin, the density of the thermoplastic resin is significantly different from that of a filler, and thus the content of the filler can be accurately measured as described above and as a result excellent feedback control can be performed.

While an example of producing a two-layer laminated film R2 having 3 components has been described in the above second embodiment, feedback control as in the second embodiment can be performed even in the production of a resin film having 3 or more resin layers and a resin film having 4 or more components. When the resin composition flowing through the pipe 31c in a molten state includes three or more thermoplastic resins, the content of each of the thermoplastic resins can be calculated based on the content of the filler calculated and the ratio of mixing of each of the resins previously determined for every grade.

Preferred embodiments of the present invention have been described above, but the present invention is not limited to the above embodiments and various modifications and changes are available. Examples of modifications will be described below.

### Modified Example 1

Although a recovered raw material Rz recovered from the same production line in which a formed product was produced was used as a raw material of the formed product in each of the above embodiments, the raw material is not limited thereto. A recovered raw material Rz recovered from another production line may also be used as a raw material of the formed product. Furthermore, a recovered raw material recovered from other formed products prepared using a different type of a thermoplastic resin or formed products in which each of the content of the thermoplastic resin is different may also be used.

### Modified Example 2

In the above second embodiment, a recovered raw material Rz may also be used for forming not only the substrate layer r1 but also the surface layer r2. Feedback control similar to that for the substrate layer r1 enables the content of the filler and the thermoplastic resin in the surface layer r2 to be easily kept in a certain range also in this case.

### Modified Example 3

Instead of supplying chips of the recovered raw material Rz prepared by crushing after discharge to the extruder 31, pellets previously prepared using the chips may be supplied to the extruder 31.

Alternatively, the recovered raw material Rz may be previously pelletized together with a new raw material. By previously pelletizing all of the raw materials and supplying them into the extruder 31, the composition of the raw materials of the film to be produced is more likely to be stable.

Pellets have a size of preferably 1 mm or more, and more preferably 2 mm or more, and preferably 10 mm or less and more preferably 6 mm or less from the viewpoint of handling in melt-kneading.

### Modified Example 4

For more stable control, a radiation meter such as the measuring device 5 may be provided in the line for the recovered raw material Rz to calculate the content of the filler in the recovered raw material Rz. Furthermore, no measuring device 5 may be provided in the line for the substrate layer r1 and a measuring device 5 may be provided only in the line for the recovered raw material Rz to perform the above feedback control. The time to detect accidents is shorter in the case where a measuring device 5 is provided in the line for the substrate layer r1 than in the case where a measuring device 5 is provided in the line for the recovered raw material Rz. Thus, it is preferable that the measuring device 5 is provided in the line for the substrate layer r1 as in the above embodiment from the viewpoint of stable control.

### Modified Example 5

The resin film described above is only an example of formed products produced in the present invention. The present invention may also be applied to the production of formed products such as pellets and containers as long as the resin composition is formed.

Pellets may be formed by extruding a resin composition which has been melted as described above into strands and by cutting them. Methods such as strand cut, under water cut and hot cut may be used.

Examples of methods of forming containers include injection forming, blow forming and in-mold forming.

The step of melt-kneading a resin composition and extruding it is the same regardless of the shape of formed products. Thus, even in the production of formed products having a different shape, the amount of raw materials supplied can be feedback-controlled so that the content of the filler in the formed product reaches the target value by measuring the density of the resin composition in a molten state in the pipe 31c of the extruder 31 as in the production of a resin film. This ensures a certain quality of the formed product to be produced for long time.

### Modified Example 6

The target to be measured in the measuring device 5 may not always be in a molten state, and may be in the form of solid such as a solid sheet, a structure or pellets. The content of raw materials in solid can also be calculated using radiation in the same manner as in a molten state. However, the conditions of measurement always need to be kept constant, and thus it is preferable that the target of measurement is in a molten state considering that the conditions of measurement can be easily kept constant.

### Other Modified Examples

In the case of a laminated film R2, a recovered raw material Rz may also be recovered from a monolayer film of the substrate layer r1 or the surface layer r2 before lamination. The above feedback control is also effective even when a recovered raw material Rz in which the content of the filler is different is mixed as described above.

Although the density and the content of the filler are calculated in the calculator 54 in each of the embodiments, they may be calculated in the controller 6. Furthermore, while the calculator 54 is provided outside the measuring device 5, it may be provided in the measuring device 5.

The method of forming a film of a resin composition is not limited to the above extrusion (cast forming) using a die 31b. The present invention may also be applied to other forming methods such as inflation forming using an O die and calendering using a pressure roll.

While an example in which the surface layer r2 is laminated on the substrate layer r1 by extrusion lamination has been described in the second embodiment, the present invention may also be applied to lamination by other methods such as coextrusion, film lamination and coating.

Furthermore, the resin film may be an unstretched film or a stretched film. The resin film is preferably a stretched film from the viewpoint of formability of pores. When the formed product in the present invention is a resin film, the resin film is used for various purposes such as printing paper, wrapping paper and wallpaper.

While the content of filler was calculated out of a plurality of raw materials in the above embodiments, the content of each resin in a resin film in which a plurality of resins are mixed in a sea-island structure without filler may also be calculated as long as the resins have a different density. For example, polypropylene has a density of about 0.7 g/cm³ at 230° C. and polyethylene terephthalate has a density of about 1.0 to 1.1 g/cm³ at 230° C. The content of raw materials in such a resin composition including a plurality of resins with a different density can be controlled by applying the present invention.

The present application claims priority to Japanese Patent Application No. 2020-6614 filed Jan. 20, 2020, the content of which is incorporated herein by reference.

### REFERENCE SINGS LIST

1, 2 System for producing formed product, 21a to 21c Meters, 31, 311, 312 Extruder, 5 Measuring device, 51 Detecting member, 54 Calculator, 6 Controller

1. A method for producing a formed product using a resin composition comprising a plurality of raw materials, the method comprising:
supplying each of the plurality of raw materials to an extruder;
melt-kneading the plurality of raw materials in the extruder to prepare a resin composition; and
irradiating the prepared resin composition with radiation and calculating the content of the raw material in the resin composition based on the results of detection of the radiation transmitted through the resin composition.

2. The method according to claim 1, wherein
the plurality of raw materials comprise at least a thermoplastic resin and a filler, and
the calculating the content comprises calculating the content of the filler in the resin composition.

3. The method according to claim 2, wherein
the filler is an inorganic filler.

4. The method according to claim 1, wherein the calculating the content comprises
irradiating the resin composition in a molten state with radiation and detecting the radiation transmitted through the resin composition.

5. The method according to claim 1, wherein the calculating the content comprises:
calculating the density of the resin composition from the results of detection of the radiation, and
calculating the content of the raw material based on the calculated density of the resin composition and the density of each of the raw materials in the resin composition.

6. The method according to claim 1, wherein
the calculating the content comprises calculating the content of the raw material based on at least one condition of the temperature and the pressure of the resin composition upon irradiation with the radiation.

7. The method according to claim 1, wherein
the radiation is an X ray or a γ ray.

8. The method according to claim 1, wherein
the formed product is continuously produced.

9. The method according to claim 1, wherein
part of the plurality of raw materials supplied to the extruder is supplied to the extruder in the form of a mixture.

10. The method according to claim 1, wherein
the mixture is a recovered raw material comprising the whole or part of the formed product discharged in a process of producing the formed product.

11. The method according to claim 1, comprising
calculating the content of a raw material in the recovered raw material supplied to the extruder based on the content of the raw material in the resin composition and the amount of each of the raw materials supplied.

12. The method according to claim 1, wherein
the formed product is a film or a pellet.

13. The method according to claim 1, comprising
controlling the amount of each of the raw materials supplied to the extruder based on the calculated content of the raw material.

14. A system for producing a formed product using a resin composition comprising a plurality of raw materials, the system comprising:
an extruder for melt-kneading the plurality of raw materials to prepare a resin composition;
a measuring device for irradiating the prepared resin composition with radiation and measuring the results of detection of the radiation transmitted through the resin composition; and
a calculator for calculating the content of the raw material in the resin composition based on the results of detection.

15. The system according to claim 14, comprising
a controller for controlling the amount of each of the raw materials supplied to the extruder based on the calculated content of the raw material.
